(19) United States
(12) Patent Application Publication
(10) Pub. No.: US 2009/0209601 A1
(43) Pub. Date: Aug. 20, 2009

(54) USE OF RXR AGONISTS FOR THE TREATMENT OF OSTEOARTHRITIS

(75) Inventors: Sunil Nagpal, Collegeville, PA (US); Zhiyong Yang, Newton, MA (US); Elisabeth Morris, Sherborn, MA (US); Edward R. Lavallie, Harvard, MA (US); Lisa A. Collins-Racie, Acton, MA (US)

Correspondence Address: POTTER ANDERSON & CORROON LLP/WYETH;ATTN: JANET E. REED, PH.D., 1313 NORTH MARKET STREET, HERCULES PLAZA, SIXTH FLOOR, WILMINGTON, DE, 19801, US

(73) Assignee: Wyeth, Madison, NJ (US)

(21) Appl. No.: 12/369,425

(22) Filed: Feb. 11, 2009

Related U.S. Application Data
(60) Provisional application No. 61/069,919, filed on Mar. 19, 2008.
(60) Provisional application No. 61/065,953, filed on Feb. 15, 2008.

(51) Int. Cl. A61K 31/44; A61K 31/203; A61K 31/202; A61K 31/20; C12Q 1/68; A61P 19/02

(52) U.S. Cl. 514/356; 514/559; 514/560; 514/558; 435/6

(57) ABSTRACT

Disclosed herein are methods of preventing and treating osteoarthritis through the use of RXR agonists.

### CROSS-REFERENCE TO RELATED APPLICATIONS

This application claims the benefit of U.S. Provisional Application No. 61/069,919, filed Mar. 19, 2008, and U.S. Provisional Application No. 61/065,953, filed Feb. 15, 2008, both of which are incorporated by reference in their entireties.

### FIELD OF THE INVENTION

The present invention relates to methods of treating or preventing osteoarthritis with RXR agonists.

### BACKGROUND OF THE INVENTION

Osteoarthritis, also known as degenerative joint disease, is characterized by degeneration of articular cartilage as well as proliferation and remodeling of subchondral bone. The usual symptoms are stiffness, limitation of motion, and pain. Osteoarthritis is the most common form of arthritis, and prevalence rates increase markedly with age.

Existing osteoarthritis treatment approaches include exercise, medicines, rest and joint care, surgery, pain relief techniques, alternative therapies, and weight control. The commonly used medicines in treating osteoarthritis include nonsteroidal anti-inflammatory drugs (NSAIDs), for example, aspirin, ibuprofen, naproxen sodium, ketoprofen; topical pain-relieving creams, rubs, and sprays (for example, capsaicin cream) applied directly to the skin; corticosteroids, typically injected into affected joints to relieve pain temporarily; and hyaluronic acid. Surgery may be performed to resurface (smooth out) bones, reposition bones, and replace joints. Although various medications have been used for treating the disease, they are not effective for long term control and prevention.

Retinoid X receptors (RXRs) are members of a large superfamily of intracellular hormone receptors. These proteins bind to specific DNA sequences and directly regulate transcription of target genes in response to activation by their specific ligands (Leid et al., Trends Biochem. Sci. 17:427-33 (1992); Leid et al., Cell 68:377-95 (1992); Mangelsdorf et al., Nature 345:224-29 (1990); and Yu et al., Cell 67:1251-66 (1991)). The RXRs belong to a large subgroup of the superfamily defined by a conserved subregion within the DNA binding domain. This group also includes the receptors for retinoic acid, thyroid hormone, and vitamin D as well as a number of other less well characterized proteins, called orphan receptors, that do not have known ligands. As monomers, the members of this class can bind to sequences related to the hexameric consensus AGGTCA. RXR homodimers bind to tandem repeats of this consensus separated by a single base pair (Manglesdorf et al., Cell 66:555-61 (1991)), and apparently to additional elements including β-RARE (Zhang et al., Nature 358:587-91 (1992)). These homodimer binding sites confer specific response to 9-cis retinoic acid (9-cis-RA), the ligand for the RXRs. In addition, the RXRs heterodimerize with a variety of other family members, including the receptors for all-trans-retinoic acid, thyroid hormone (T3), and vitamin D. This heterodimerization strongly increases the affinity of these receptors for their specific response elements (Yu et al., supra; Zhang et al., supra; Bugge et al., EMBO J. 11:1409-18 (1992)), and recent evidence also demonstrates that it is also required for full hormone dependent transcriptional activity of at least the thyroid hormone receptor-RXR complex.

Mammals have three genes encoding alpha, beta, and gamma isoforms of RXR (Mangelsdorf et al., Genes Dev. 6:329-44 (1992)). The expression patterns of murine RXRs (Mangelsdorf et al. (1992), supra) and homologues of RXR found in Xenopus (Blumberg et al., Proc. Natl. Acad. Sci. USA 89:2321-25, (1992)) and Drosophila (Oro et al., Nature 347:298-301 (1990)) suggest that the members of the RXR family play important roles in several aspects of development and central nervous system differentiation as well as in adult physiology. Based on both their specific response to the 9-cis-RA metabolite and their heterodimerization with the RARs, it is clear that the RXRs play a central role in the broad regulatory effects of retinoids. Moreover, their heterodimeric interactions with other family members indicate that the RXRs also play a central role in response to thyroid hormone, vitamin D, and perhaps other compounds. This dual function is unique within the nuclear receptor superfamily.

Liver X receptors (LXRs), originally identified from liver as orphan receptors, are members of the nuclear hormone receptor super family and have been found to be negative regulators of macrophage inflammatory gene expression (see Published U.S. Patent Application No. 2004/0259948; Joseph S B et al., Nat. Med. 9:213-19 (2003)). LXRs are ligand-activated transcription factors and bind to DNA as obligate heterodimers with retinoid X receptors. While LXRα is restricted to certain tissues such as liver, kidney, adipose, intestine, and macrophages, LXRβ displays a ubiquitous tissue distribution pattern. Activation of LXRs by oxysterols (endogenous ligands) in macrophages results in the expression of several genes involved in lipid metabolism and reverse cholesterol transport, including ABCA1, ABCG1, and apolipoprotein E.

### SUMMARY OF THE INVENTION

One aspect is for a method for the treatment of a mammal suffering from osteoarthritis comprising administering to the mammal in need thereof an RXR-responsive gene expression-modulating amount of an RXR agonist.

Another aspect is for a method for the treatment of a mammal suffering from osteoarthritis comprising administering to the mammal in need thereof an effective amount of an RXR agonist to relieve pain in osteoarthritic joints.

A further aspect is for a method of indentifying an RXR ligand capable of reducing an osteoarthritic effect in cartilage comprising: (a) providing a sample containing RXR; (b) contacting the sample with a test compound; and (c) determining whether the test compound reduces an osteoarthritic effect in cartilage.

Other aspects and advantages of the present invention will become apparent to those skilled in the art upon reference to the detailed description that hereinafter follows.

### BRIEF DESCRIPTION OF THE FIGURES

FIG. 1. Expression of selected human nuclear receptors in articular cartilage from subjects with osteoarthritis compared to normal cartilage. mRNA levels for nuclear receptors judged to be expressed (“present”) in HG-U95Av2 Affymetrix GeneChip® data of articular cartilage from severe OA patients. Values on the Y-axis reflect transcript levels measured on GeneChips® and expressed in parts per million (ppm). LXR: liver X receptor; RXR: retinoid X receptor; RAR: retinoic acid receptor; Rev: Rev-erb; GR: glucocorticoid receptor; EAR: v-erbA-related; COU: chicken ovalbumin upstream promoter transcription factor; CAR: constitutive androstane receptor; PXR: pregnane X receptor; MR: mineralocorticoid receptor; SF: steroidogenic factor; TR: thyroid hormone receptor; NOR: neuron-derived orphan receptor; Nurr: Nur-related; SHP: small heterodimer partner; FXR: farnesoid X receptor.

FIG. 2. Quantitative RT-PCR for LXRα (A), LXRβ (B), RXRα (C), and RARγ (D) was performed on matched non-lesional (M) and lesional (S) cartilage samples from two human OA donors (83 and 86), and were compared to cartilage samples from two normal donors (Control 1 and 2). Bars represent the mean of replicate qRT-PCR reactions ±SEM * p<0.05, ** p<0.01, comparison of all OA samples to normal samples; # p<0.05, ## p<0.01, comparison of lesional cartilage samples to normals; [symbol] p<0.01, comparison of non-lesional cartilage samples to normals, or non-lesional cartilage samples to matched lesional cartilage samples, as indicated by brackets in the figure.

FIG. 3. Comparison of RXRα and RXRβ nuclear receptor expression in non-lesional and lesional human osteoarthritic articular cartilage compared to normal cartilage. Nuclear receptor expression data from normal (n=10; white bars), non-lesional OA cartilage (n=10; gray bars), and lesional OA cartilage (n=10; black bars) RNA samples using the human NR-TLDA, expressed as mean RQ (fold-change) ±SEM for that cohort compared to normal sample Control 1 following normalization to the GUSB (β-glucuronidase) endogenous control. ** p<0.05 by Welch t test for both lesional OA vs. normal and non-lesional OA vs. normal comparisons.

FIG. 4. Primary OA chondrocytes down regulate RXRα and RXRγ in response to treatment with IL-1β or TNFα. Primary chondrocytes isolated from human donors (OA n=2, light gray and dark gray bars; normal n=2, white and black bars) were treated in monolayer culture with either 1 ng/mL IL-1 or 10 ng/mL TNFα for 18 hours in triplicate cultures per treatment. RNA prepared from the cells following culture was assayed by qRT-PCR to measure the effect of cytokine treatment on the expression of (A) RXRα and (B) RXRβ. Bars represent the individual average fold change in expression values for the cytokine-treated cultures for each donor compared to untreated cultures from the same donor, ±SD. **p<0.01 vs. control by Welch t test.

### DETAILED DESCRIPTION OF THE INVENTION

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning: A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); U.S. Pat. No. 4,683,195; Nucleic Acid Hybridization (B. D. Hames & S. J. Higgins eds. 1984); Transcription and Translation (B. D. Hames & S. J. Higgins eds. 1984); Culture of Animal Cells (R. I. Freshney, Alan R. Liss, Inc., 1987); Immobilized Cells and Enzymes (IRL Press, 1986); B. Perbal, A Practical Guide to Molecular Cloning (1984); Methods in Enzymology (Academic Press, Inc., N.Y.); Gene Transfer Vectors for Mammalian Cells (J. H. Miller and M. P. Calos eds., 1987, Cold Spring Harbor Laboratory); Methods in Enzymology, Vols. 154 and 155 (Wu et al. eds.), Immunochemical Methods in Cell and Molecular Biology (Mayer and Walker, eds., Academic Press, London, 1987); Handbook of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Here, Applicants show that RXRα and RXRβ are expressed in normal, non-lesional osteoarthritic, and lesional (severe) osteoarthritic cartilages. In addition, Applicants find that the transcriptional level of RXRα and RXRβ are significantly decreased in cartilage from osteoarthritis patients compared to normals. Furthermore, Applicants find that RXRγ is also expressed in articular cartilage, and the expression of RXRα and RXRγ in articular chondrocytes is significantly reduced by inflammatory cytokines II-1β (RXRα) and TNFα (RXRγ). The impact of dysregulated RXR expression in OA cartilage is expected to be pleiotropic, since RXR isoforms can dimerize with each other, or they can heterodimerize with several other nuclear receptors including LXRs (reviewed in Germain et al., Pharmacol. Rev. 58:760-72 (2006)). RXR biology is further complicated by the fact that some heterodimeric receptor complexes (e.g. LXRs, FXR, and PPARs) can be independently activated by either the RXR's ligand, the RXR partner's ligand, or by both; alternatively, other RXR heterodimeric receptor complexes require the partner's ligand for activation (e.g. VDR, and TR). Applicants have previously shown the importance of LXR signaling in OA cartilage (Published U.S. Patent Application No. 2009/0012053), and the potentially destructive consequences of an LXR signaling deficit in OA cartilage. Since RXRs are obligate heterodimers for LXRs, then the decrease in RXR expression that Applicants have discovered in OA cartilage may account for some or all of the observed decrease in LXR activity in the disease tissue. In addition, RXRs partner with other nuclear receptors (such as VDR and PPAR) that are expressed in cartilage and may be important for cartilage homeostasis; therefore, a reduction in RXR expression and activity may negatively impact those signaling pathways as well.

### I. Definitions

In the context of this disclosure, a number of terms shall be utilized.

As used herein, the term “about” or “approximately” means within 20%, preferably within 10%, and more preferably within 5% of a given value or range.

The terms “effective amount”, “therapeutically effective amount”, “an RXR-responsive gene expression-inducing amount”, and “effective dosage” as used herein, refer to the amount of an effector molecule that, when administered to a mammal in need, is effective to at least partially ameliorate or to at least partially prevent conditions related to osteoarthritis.

As used herein, the term “expression” includes the process by which DNA is transcribed into mRNA and translated into polypeptides or proteins.

“Retinoid X Receptor” or “RXR” refers to RXRα, RXRβ, and RXRγ, and variants isoforms, and active fragments thereof. RXRβ is ubiquitously expressed, while RXRα expression is limited to liver, kidney, spleen, placenta, epidermis, and, as demonstrated herein, cartilage. RXRγ is expressed in muscle and brain, and, as demonstrated herein, cartilage. Representative GenBank® accession numbers for RXRα sequences include the following: human (Homo sapiens, NP_{—}002948), mouse (Mus musculus, NP_{—}035435, AAB36777, MB36778), rat (Rattus norvegicus, NP_{—}036937), orangutan (Pongo abelii, NP_{—}001125717), zebrafish (Danio rerio, NP_{—}571228, A2T929), frog (Xenopus laevis, P51128). Representative GenBank® accession numbers for RXRβ sequences include the following: human (Homo sapiens, NP_{—}068811), mouse (Mus musculus, NP_{—}035436, BAA04859), rat (Rattus norvegicus, NP_{—}996731), cow (Bos taurus, NP_{—}001077109), frog (Xenopus laevis, NP_{—}001080936, NP_{—}001081830), zebrafish (Danio rerio, NP_{—}571350, NP_{—}571313, Q90415), dog (Canis lupus familiaris, Q5TJF7). Representative GenBank® accession numbers for RXRγ sequences include the following: human (Homo sapiens, NP_{—}008848, NP_{—}001009598), mouse (Mus musculus, NP_{—}033133), rat (Rattus norvegicus, NP_{—}113953), cow (Bos taurus, NP_{—}001068876), chicken (Gallus gallus, NP_{—}990625), zebrafish (Danio rerio, NP_{—}571292, Q6DHP9), orangutan (Pongo abelii, NP_{—}001124824), pig (Sus scrofa, NP_{—}001123685), frog (Xenopus laevis, P51129).

“Liver X receptor” or “LXR” refers to both LXRα and LXRβ, and variants, isoforms, and active fragments thereof. LXRβ is ubiquitously expressed, while LXRα expression is limited to liver, kidney, intestine, spleen, adipose tissue, macrophages, skeletal muscle, and, as demonstrated herein, cartilage. Representative GenBank® accession numbers for LXRα sequences include the following: human (Homo sapiens, NP_{—}005684, NP_{—}001123573, NP_{—}001123574), mouse (Mus musculus, NP_{—}038867), rat (Rattus norvegicus, NP_{—}113815), cow (Bos taurus, NP_{—}001014861), pig (Sus scrofa, NP_{—}001095284), chicken (Gallus gallus, NP_{—}989873). Representative GenBank® accession numbers for LXRβ include the following: human (Homo sapiens, NP_{—}009052), mouse (Mus musculus, NP_{—}033499), rat (Rattus norvegicus, Q62755), cow (Bos taurus, Q5BIS6).

The term “mammal” refers to a human, a non-human primate, canine, feline, bovine, ovine, porcine, murine, or other veterinary or laboratory mammal. Those skilled in the art recognize that a therapy which reduces the severity of a pathology in one species of mammal is predictive of the effect of the therapy on another species of mammal.

The term “modulate” encompasses either a decrease or an increase in activity or expression depending on the target molecule. For example, an RXRα modulator is considered to modulate the expression or activity of RXRα if the presence of such RXRα modulator results in an increase or decrease in RXRα expression or activity.

### II. RXR and LXR Agonists

RXR agonists useful in the present invention include, but are not limited to, compounds that preferentially activate RXR over RAR (i.e. RXR specific agonists) and compounds that activate both RXR and RAR (i.e. pan agonists). It also includes compounds that activate RXR in a certain cellular context but not others (i.e. partial agonists). Representative compounds include those disclosed in U.S. Pat. Nos. 5,399,586, 5,466,861, 5,801,253, 6,506,917, 5,780,676, 5,962,731, 6,320,074, 5,972,881, 5,770,378, and 5,721,103, and in Boehm et al., J. Med. Chem. 38:3146-55 (1995), Boehm et al., J. Med. Chem. 37:2930-41 (1994), Antras et al., J. Biol. Chem. 266:1157-61 (1991), Salazar-Olivo et al., Biochem. Biophys. Res. Commun. 204:257-63 (1994), and Safanova, Mol. Cell. Endocrin. 104:201-11 (1994). Pan agonists include, but are not limited to, 9-cis retinoic acid, docosahexanoic acid, and phytanic acid. Useful synthetic agonists include LG100268 (6-[1-(3,5,5,8,8-pentamethyl-5,6,7,8-tetrahydronaphthalen-2-yl)cyclopropyl]pyridine-3-carboxylic acid) and bexarotene(4-[1-(5,6,7,8-tetrahydro-3,5,5,8,8-pentamethyl-2-naphthalenyl) ethenyl]benzoic acid).

LXR agonists useful in the present invention include natural oxysterols, synthetic oxysterols, synthetic nonoxysterols, and natural nonoxysterols. Exemplary natural oxysterols include 20(S) hydroxycholesterol, 22(R) hydroxycholesterol, 24(S) hydroxycholesterol, 25-hydroxycholesterol, 24(S),25 epoxycholesterol, and 27-hydroxycholesterol. Exemplary synthetic oxysterols include N,N-dimethyl-3β-hydroxycholenamide (DMHCA). Exemplary synthetic nonoxysterols include N-(2,2,2-trifluoroethyl)-N-{4-[2,2,2-trifluoro-1-hydroxy-1-(trifluoromethyl)ethyl]phenyl}benzene sulfonamide (TO901317; Tularik 0901317), [3-(3-(2-chloro-trifluoromethylbenzyl-2,2-diphenylethylamino)propoxy)phenylacetic acid] (GW3965), N-methyl-N-[4-(2,2,2-trifluoro-1-hydroxy-1-trifluoromethyl-1-ethyl)-phenyl]-benzenesulfonamide (TO314407), 4,5-dihydro-1-(3-(3-trifluoromethyl-7-propyl-benzisoxazol-6-yloxy)propyl)-2,6-pyrimidinedione, 3-chloro-4-(3-(7-propyl-3-trifluoromethyl-6-(4,5)-isoxazolyl)propylthio)-phenyl acetic acid (F₃MethylAA), and acetyl-podocarpic dimer. Exemplary natural nonoxysterols include paxilline, desmosterol, and stigmasterol.

Other useful LXR agonists are disclosed, for example, in Published U.S. Patent Application Nos. 2006/0030612, 2005/0131014, 2005/0036992, 2005/0080111, 2003/0181420, 2003/0086923, 2003/0207898, 2004/0110947, 2004/0087632, 2005/0009837, 2004/0048920, and 2005/0123580; U.S. Pat. Nos. 6,316,503, 6,828,446, 6,822,120, and 6,900,244; WO01/41704; Menke J G et al., Endocrinology 143:2548-58 (2002); Joseph S B et al., Proc. Natl. Acad. Sci. USA 99:7604-09 (2002); Fu X et al., J. Biol. Chem. 276:38378-87 (2001); Schultz J R et al., Genes Dev. 14:2831-38 (2000); Sparrow C P et al., J. Biol. Chem. 277:10021-27 (2002); Yang C et al., J. Biol. Chem. 281:27816-26 (2006); Bramleft K S et al., J. Pharmacol. Exp. Ther. 307:291-96 (2003); Ondeyka J G et al., J. Antibiot (Tokyo) 58:559-65 (2005).

### III. Methods of Treatment/Prevention

According to one modulatory method, RXR activity is stimulated in a cell by contacting the cell with an RXR agonist. Examples of such RXR agonists are described above in Section II. Other RXR agonists that can be used to stimulate the RXR activity can be identified using screening assays that select for such compounds, as described in detail herein (Section V).

Modulatory methods can be performed in vitro (e.g., by culturing the cell with an RXR agonist or by introducing an RXR agonist into cells in culture) or, alternatively, in vivo (e.g., by administering an RXR agonist to a subject or by introducing an RXR agonist into cells of a subject). For practicing a modulatory method in vitro, cells can be obtained from a subject by standard methods and incubated (i.e., cultured) in vitro with an RXR agonist to modulate RXR activity in the cells.

### 1. Prophylactic Methods

In one aspect, the invention provides a method for preventing osteoarthritis in a subject by administering to the subject an RXR agonist. Administration of a prophylactic RXR agonist can occur prior to the manifestation of osteoarthritis symptoms, such that osteoarthritis is prevented or, alternatively, delayed in its progression.

2. Therapeutic Methods Another aspect of the invention pertains to methods of modulating RXR activity for osteoarthritis therapeutic purposes. Accordingly, in an exemplary embodiment, a modulatory method of the invention involves contacting a cell with an RXR agonist. These modulatory methods can be performed in vitro (e.g., by culturing the cell with an RXR agonist) or, alternatively, in vivo (e.g., by administering an RXR agonist to a subject).

RXR agonists can also be useful for treating pain in osteoarthritic joints. For example, RXR agonists can be effective in treating acute pain (short duration) or chronic pain (regularly reoccurring or persistent) associated with osteoarthritis.

### IV. Administration of RXR Agonists

RXR agonists are administered to subjects in a biologically compatible form suitable for pharmaceutical administration in vivo. By “biologically compatible form suitable for administration in vivo” is meant a form of the RXR agonist to be administered in which any toxic effects are outweighed by the therapeutic effects of the agonist. The term “subject” is intended to include living organisms in which an immune response can be elicited, for example, mammals. Administration of RXR agonists as described herein can be in any pharmacological form including a therapeutically effective amount of an RXR agonist alone or in combination with a pharmaceutically acceptable carrier.

A therapeutically effective amount of an RXR agonist may vary according to factors such as the disease state, age, sex, and weight of the individual, and the ability of the RXR agonist to elicit a desired response in the individual. Dosage regime may be adjusted to provide the optimum therapeutic response. For example, several divided doses may be administered daily, or the dose may be proportionally reduced as indicated by the exigencies of the therapeutic situation.

The therapeutic or pharmaceutical compositions of the present invention can be administered by any suitable route known in the art including, for example, oral, intravenous, subcutaneous, intramuscular, transdermal, intrathecal, or intracerebral or administration to cells in ex vivo treatment protocols. Administration can be either rapid as by injection or over a period of time as by slow infusion or administration of slow release formulation. For treating or preventing osteoarthritis, administration of the therapeutic or pharmaceutical compositions of the present invention can be performed, for example, by oral administration or by intra-articular injection.

Furthermore, RXR agonists can be stably linked to a polymer such as polyethylene glycol to obtain desirable properties of solubility, stability, half-life, and other pharmaceutically advantageous properties (see, e.g., Davis et al., Enzyme Eng. 4:169-73 (1978); Burnham N L, Am. J. Hosp. Pharm. 51:210-18 (1994)).

RXR agonists can be in a composition that aids in delivery into the cytosol of a cell. For example, an RXR agonist may be conjugated with a carrier moiety such as a liposome that is capable of delivering the agonist into the cytosol of a cell. Such methods are well known in the art (see, e.g., Amselem S et al., Chem. Phys. Lipids 64:219-37 (1993)). In addition, an RXR agonist can be delivered directly into a cell by microinjection.

RXR agonists can be employed in the form of pharmaceutical preparations. Such preparations are made in a manner well known in the pharmaceutical art. One preferred preparation utilizes a vehicle of physiological saline solution, but it is contemplated that other pharmaceutically acceptable carriers such as physiological concentrations of other non-toxic salts, five percent aqueous glucose solution, sterile water or the like may also be used. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the RXR agonist, use thereof in the therapeutic compositions is contemplated. Supplementary active compounds can also be incorporated into the compositions. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous. RXR agonists can also be incorporated into a solid or semi-solid biologically compatible matrix which can be implanted into tissues requiring treatment.

The carrier can also contain other pharmaceutically-acceptable excipients for modifying or maintaining the pH, osmolarity, viscosity, clarity, color, sterility, stability, rate of dissolution, or odor of the formulation.

Dose administration can be repeated depending upon the pharmacokinetic parameters of the dosage formulation and the route of administration used.

It is also provided that certain formulations containing RXR agonists are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents, or flavoring agents. The compositions may be formulated so as to provide rapid, sustained, or delayed release of the active ingredients after administration to the patient by employing procedures well known in the art. The formulations can also contain substances that diminish proteolytic degradation and/or substances which promote absorption such as, for example, surface active agents.

It is especially advantageous to formulate compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the RXR agonist and the particular therapeutic effect to be achieved and (b) the limitations inherent in the art of compounding such an active compound for the treatment of OA in individuals. The specific dose can be readily calculated by one of ordinary skill in the art, e.g., according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the RXR agonist activities disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated; the choice of composition to be administered; the age, weight, and response of the individual patient; the severity of the patient's symptoms; and the chosen route of administration.

Toxicity and therapeutic efficacy of such RXR agonists can be determined by standard pharmaceutical procedures in cell cultures or experimental animals, for example, for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. RXR agonists that exhibit large therapeutic indices are preferred. While RXR agonists that exhibit toxic side effects may be used, care should be taken to design a delivery system that targets such agonists to the site of affected tissue in order to minimize potential damage to uninfected cells and, thereby, reduce side effects.

The data obtained from the cell culture assays and animal studies can be used in formulating a range of dosage for use in humans. The dosage of such RXR agonists lies preferably within a range of circulating concentrations that include the ED₅₀ with little or no toxicity. The dosage may vary within this range depending upon the dosage form employed and the route of administration utilized. For any RXR agonist used in a method of the invention, the therapeutically effective dose can be estimated initially from cell culture assays. A dose may be formulated in animal models to achieve a circulating plasma concentration range that includes the IC₅₀ (i.e., the concentration of RXR agonist that achieves a half-maximal inhibition of symptoms) as determined in cell culture. Such information can be used to more accurately determine useful doses in humans. Levels in plasma may be measured, for example, by high performance liquid chromatography.

Monitoring the influence of RXR agonists can be applied not only in basic drug screening, but also in clinical trials. To study the effect of RXR agonists on osteoarthritis, for example, in a clinical trial, articular chondrocytes can be isolated and RNA prepared and analyzed for the levels of expression of TNFα and other genes implicated in osteoarthritis. The levels of gene expression (i.e., a gene expression pattern) can be quantified by Northern blot analysis or RT-PCR, by measuring the amount of protein produced, or by measuring the levels of activity of genes, all by methods well known to those of ordinary skill in the art. In this way, the gene expression pattern can serve as a marker, indicative of the physiological response of the cells to the RXR agonist. Accordingly, this response state may be determined before, and at various points during, treatment of the individual with the RXR agonist.

Furthermore, in the treatment of osteoarthritis, compositions containing RXR agonists can be administered exogenously, and it would likely be desirable to achieve certain target levels of RXR agonist in sera, in any desired tissue compartment, and/or in the affected tissue. It would, therefore, be advantageous to be able to monitor the levels of RXR agonist in a patient or in a biological sample including a tissue biopsy sample obtained from a patient. Accordingly, the present invention also provides methods for detecting the presence of RXR agonist in a sample from a patient.

### V. Screening Assays

In one embodiment, expression levels of RXR-responsive genes or activity levels of proteins therefrom can be used to facilitate design and/or identification of compounds that treat osteoarthritis through an RXR-based mechanism. Accordingly, the invention provides methods (also referred to herein as “screening assays”) for identifying RXR agonists. Compounds thus identified can be used in the treatment of osteoarthritis as described elsewhere herein.

Test compounds can be obtained, for example, using any of the numerous approaches in combinatorial library methods known in the art, including spatially addressable parallel solid phase or solution phase libraries; synthetic library methods requiring deconvolution; the ‘one-bead one-compound’ library method; and synthetic library methods using affinity chromatography selection.

Examples of methods for the synthesis of molecular libraries can be found in, for example: DeWitt S H et al., Proc. Natl. Acad. Sci. U.S.A. 90:6909-13 (1993); Erb E et al., Proc. Natl. Acad. Sci. USA 91:11422-26 (1994); Zuckermann R N et al., J. Med. Chem. 37:2678-85 (1994); Cho C Y et al., Science 261:1303-05 (1993); Carrell et al., Angew. Chem. Int. Ed. Engl. 33:2059 (1994); Carrell et al., Angew. Chem. Int. Ed. Engl. 33:2061 (1994); Gallop M A et al., J. Med. Chem. 37:1233-51 (1994).

Libraries of compounds may be presented in solution (e.g., Houghten R A et al., Biotechniques 13:412-21 (1992)), or on beads (Houghten R A et al., Nature 354:82-84 (1991)), chips (Fodor S A et al., Nature 364:555-56 (1993)), bacteria (U.S. Pat. No. 5,223,409), spores (U.S. Pat. No. 5,223,409), plasmids (Cull M G et al., Proc. Natl. Acad. Sci. USA 89:1865-69 (1992)) or on phage (Scott J K & Smith G P, Science 249:386-90 (1990); Devlin J J et al., Science 249:404-06 (1990); Cwirla S E et al., Proc. Natl. Acad. Sci. 87:6378-82 (1990); Felici F et al., J. Mol. Biol. 222:301-10 (1991); U.S. Pat. No. 5,223,409.).

An exemplary screening assay is a cell-based assay in which a cell that expresses RXR is contacted with a test compound, and the ability of the test compound to treat an osteoarthritic condition through an RXR-based mechanism. Determining the ability of the test compound to treat an osteoarthritic condition can be accomplished by monitoring, for example, DNA, mRNA, or protein levels, or by measuring the levels of activity of, e.g., TNFα, all by methods well known to those of ordinary skill in the art. The cell, for example, can be of mammalian origin, e.g., human.

Novel modulators identified by the above-described screening assays can be used for treatments as described herein.

### EXAMPLES

The present invention is further defined in the following Examples. It should be understood that these Examples, while indicating preferred embodiments of the invention, are given by way of illustration only. From the above discussion and these Examples, one skilled in the art can ascertain the preferred features of this invention, and without departing from the spirit and scope thereof, can make various changes and modification of the invention to adapt it to various uses and conditions.

Data are expressed as means±standard error of the mean (SEM) unless otherwise indicated. Statistical significance was determined by two-tailed Welch test or Student's t test using either Expressionist software (Genedata, Basel, Switzerland) or Microsoft Excel 2000; for either test, p<0.05 was considered significant.

### Media, Chemicals and Reagents

All cell culture reagents were obtained from Gibco-BRL (Grand Island, N.Y.). IL-1β and TNFα were purchased from R&D Systems (Minneapolis, Minn.). Human Universal Reference Total RNA (catalog #636538) was purchased from Clontech (Mountain View, Calif.). Fresh human OA and normal articular cartilage for cell culture experiments was obtained from the National Disease Research Interchange (Philadelphia, Pa.).

### Isolation of RNA from Primary Cartilage Tissue and from Chondrocytes in Culture

RNA was isolated from human osteoarthritic articular cartilage samples obtained from patients (n=18, mean age=66.2 years, range 49-84 years) undergoing total knee replacement surgery (New England Baptist Hospital, Boston, Mass.), or from non-osteoarthritic cartilage obtained from above-knee amputations (n=10, mean age=71.6 years, range 43-100) (Clinomics, Pittsfield, Mass.). The OA cartilage samples were obtained as whole joints within 2 hours of surgery, and the articular cartilage was shaved from the joint surfaces taking great care to avoid any pannus, fibrotic tissues, subchondral bone, and other non-cartilaginous regions of the joint. Non-osteoarthritic cartilage samples were obtained from individuals without a clinical diagnosis or symptoms of OA, and the specimens were evaluated histologically to confirm the classification prior to inclusion in this study. Cartilage pieces were flash-frozen in liquid nitrogen and stored at −80° C. until processed for RNA isolation. The frozen cartilage was pulverized using a Spex Certiprep freezer mill Model 6750 at 15 Hz twice for 1 minute each under liquid nitrogen. The frozen powdered cartilage was resuspended in ice-cold 4M guanidinium isothiocyanate (GITC) (Invitrogen, Carlsbad, Calif.) containing 8.9 mM 2-mercaptoethanol (βME) and homogenized on ice with a Polytron homogenizer at maximum speed setting twice for 1 minute each time, with a 1 minute “rest” between homogenizations. The homogenate was centrifuged at 1500×g for 10 minutes and the supernatant was saved. The gelatinous pellet was resuspended in GITC/βME and homogenized a second time as described above. The pellet was then discarded, and the two resulting supernatant fractions were combined and incubated with Triton X-100 (2% final concentration) and sodium acetate (pH 5.5, 1.5M final concentration) sequentially for 15 minutes each. The samples were extracted once with an equal volume of acid phenol chloroform (pH 4.5) and twice with acid phenol (pH 4.5)/phenol (pH 7.5) chloroform mix (1:1). RNA was subsequently precipitated by the addition of isopropanol, and further purified using an RNeasy Mini Kit (Qiagen, Valencia, Calif.) according to the manufacturer's protocol. RNA quantity and purity was measured by ultraviolet absorbance at A260/A280, and RNA quality was assessed by the RNA6000 assay using the Agilent BioAnalyzer 2100 (Palo Alto, Calif.). RNA yields averaged between 5-10 mg of total RNA per gram of cartilage tissue.

For isolation of RNA from chondrocytes in monolayer culture, pellets were digested with collagenase (2.5 mg/ml, Sigma, St Louis, Mo.) and RNA was subsequently prepared using TRIzol reagent (Invitrogen) according to the manufacturer's protocol. Primary chondrocytes in monolayer culture were lysed by direct addition of TRIzol reagent followed by standard TRIzol RNA purification methodologies.

### Chondrocyte Cell Culture

Chondrocytes were isolated from fresh human articular cartilage using a standard method previously described (Heinlein et al., Endocr. Rev. 25:276-308 (2004)). Cells were cultured in 10% FBS containing DMEM/F12 growth media for 2-3 days in 12 well culture plates at a density of 1-2×10⁶ cells/well. Chondrocyte cultures were stimulated with cytokines (TNFα: 10 ng/ml; IL-1β: 1 ng/ml) for 18 hours.

### Measurement of mRNA Changes in Osteoarthritic and Normal Cartilage Using Microarrays

Gene expression changes in RNA from lesional (n=14) and adjacent non-lesional (n=13) osteoarthritic cartilage compared to non-osteoarthritic cartilage (n=10) were analyzed using the Human Genome HG-U95Av2 GeneChip® Array (Affymetrix, Santa Clara, Calif.) for expression profiling, as described previously (LaVallie et al., J. Biol. Chem. 281:24124-37 (2006)). Briefly, RNA extracted from individual articular cartilage tissue samples was converted to biotinylated cRNA and fragmented according to the Affymetrix protocol. The fragmented cRNAs were diluted in 1× MES buffer containing 100 μg/ml herring sperm DNA and 500 μg/ml acetylated BSA and denatured for 5 min at 99° C. followed immediately by 5 min at 45° C. Insoluble material was removed from the hybridization mixture by a brief centrifugation, and the hybridization mix was added to each array and incubated at 45° C. for 16 hr with continuous rotation at 60 rpm. After incubation, the hybridization mix was removed and the chips were extensively washed and stained with Streptavidin R-phycoerythrin (Molecular Probes, Eugene, Oreg.) using the GeneChip® Fluidics Station 400 following the manufacturer's specifications. The raw florescent intensity value of each transcript was measured at a resolution of 6 microns with a Hewlett-Packard Gene Array Scanner.

### cDNA Synthesis and Quantitative RT-PCR (TagMan®)

cDNA was prepared from purified RNA using the High-Capacity cDNA Archive Kit (Applied Biosystems, catalog #4322171) according to the manufacturer's instructions. Quantitative real-time PCR was performed using either human TaqMan® Gene Expression assays or TaqMan® Low Density Arrays (TLDA) from Applied Biosystems. Thermal cycling was performed using either an ABI Prism 7900 Sequence Detection System (for TLDA) or an ABI Prism 7700 Sequence Detection System (for individual TaqMan® Gene Expression Assays). RNA for TaqMan® analysis was purified from dissected and frozen cartilage tissue as described above, followed by two more rounds of phenol/chloroform extraction followed by RNeasy (Qiagen) column binding and elution. RNA was treated with DNase (Qiagen) during RNeasy column purification (as recommended by the supplier) to eliminate any contaminating genomic DNA, and following the RNA purification any residual genomic DNA was removed using DNA-free (Ambion, Austin, Tex.), following the manufacturer's instructions. Human Universal RNA (Clontech) was used to generate standard curves for each assay. Pre-designed TaqMan® probe/primer assay sets (Gene Expression Assays, Applied Biosystems) for individual qRT-PCR assessments were obtained for the following nuclear receptor genes: NR1H3 (LXRα), Hs00172885_m1; NR1H2 (LXRβ), Hs00173195_m1; NR2B1 (RXRα), HS01067640_m1; and NR1B3 (RARγ), Hs00171273_m1.

### Example 1

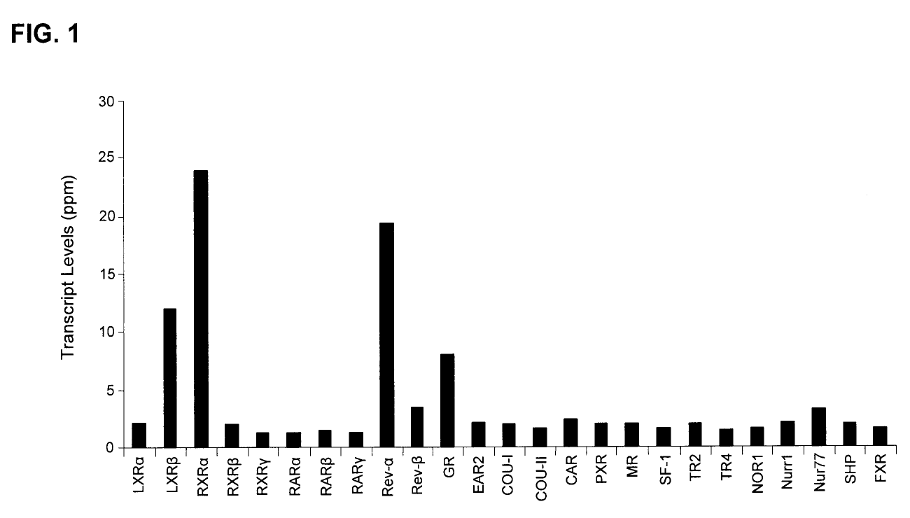

Global gene expression measurements of articular chondrocytes from lesional (n=14) and adjacent non-lesional (n=13) osteoarthritic cartilage as well as from non-osteoarthritic cartilage (n=10) using Affymetrix GeneChipe HG-U95Av.2 arrays were performed as described previously (LaVallie et al., supra). A focused analysis of these data was undertaken in an attempt to identify the spectrum of expression of nuclear hormone receptors in OA cartilage. GeneChip® software 3.2 (Affymetrix), which uses an algorithm to determine whether a gene is “present” or “absent”, as well as the specific hybridization intensity values or “average differences” of each gene on the array, was used to evaluate the gene chip data for all 49 identified human nuclear receptors (Robinson-Rechavi et al., Trends Genet. 17:554-56 (2001)). The average difference for each gene was normalized to frequency values by referral to the average differences of 11 control transcripts of known abundance that were spiked into each hybridization mix according to the procedure of Hill et al. (Science 290:809-12 (2000)). The frequency of each gene was calculated and represents a value equal to the total number of individual gene transcripts per 10⁶ total transcripts (expressed as ppm (parts per million)). Nuclear receptor transcripts that were called “present” by the GeneChip® software in at least one of the arrays for lesional OA cartilage samples were included in the analysis. The mean transcript levels of nuclear receptors represented on the gene chips and judged present in lesional OA cartilage is depicted in FIG. 1. These data confirm a previous report (Chaturvedi et al., Arthritis Rheum. 54:3513-22 (2006)) that Rev-ErbAα (“Rev-α” in FIG. 1) is one of the most abundant nuclear receptors in articular cartilage. Although the nuclear receptors listed in FIG. 1 were judged to be “present”, i.e. expressed, on the gene chips, most had transcript levels less than 5 ppm, which is below the level of reliable quantitation on the gene chips. Despite this limitation, these gene chip data indicated that LXRβ and RXRα were relatively highly expressed in articular cartilage.

### Example 2

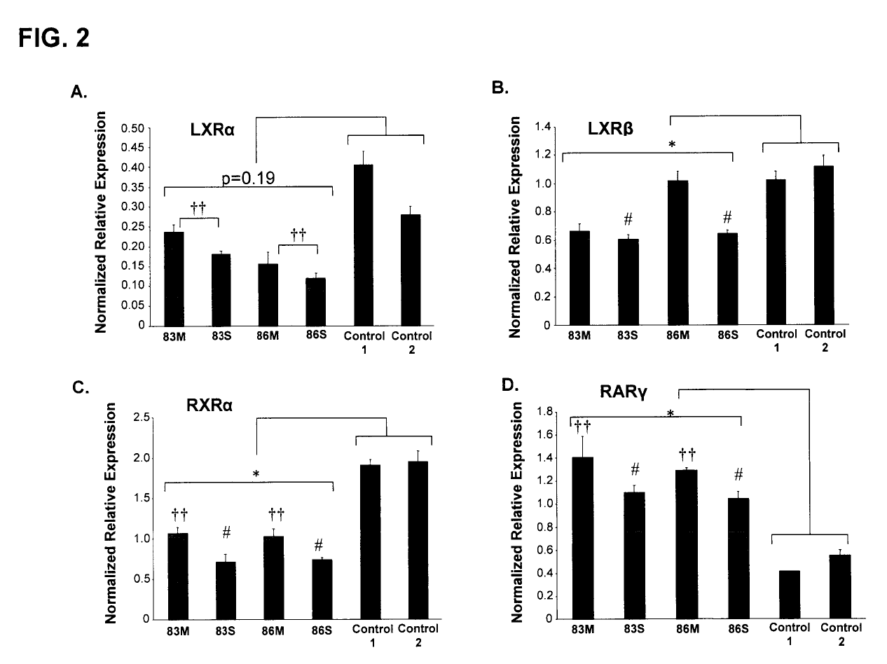

Quantitative RT-PCR experiments (qRT-PCR) were performed on cartilage RNA from a subset of the donors that were profiled by gene chip, chosen to represent cartilage with grossly severe lesions (83S and 86S), non-lesional cartilage from the same joints (83M and 86M), and cartilage from normal human joints (Control 1 and 2). Applicants measured LXRα, LXRβ, and RXRα by qRT-PCR to confirm the gene chip expression results and to investigate whether these members of LXR transcriptional complexes might be dysregulated in OA cartilage compared to normal. The results, shown in FIGS. 2A-C, confirmed that all three genes were expressed in articular cartilage. Moreover, the data showed that LXRβ and RXRα were expressed at significantly lower levels in OA cartilage samples when compared to normals (FIGS. 2B and 2C). LXRα also reflected this trend, but the data did not reach significance in this small sample set (FIG. 2A); however, a paired Student's t test revealed a significant reduction in LXRα transcript levels in lesional cartilage compared to non-lesional cartilage within donors (p=0.01). In addition, the decreased expression of LXRβ and RXRα in OA cartilage also appeared to correlate with disease severity (although the trends did not reach statistical significance in paired t tests), further supporting the possibility that LXR signaling may be compromised in OA. In contrast to the decrease in expression of RXRγ and the LXRs in OA cartilage, qRT-PCR analysis of RARγ in these same samples showed that RARγ expression was significantly increased in OA cartilage compared to normals (FIG. 2D).

### Example 3

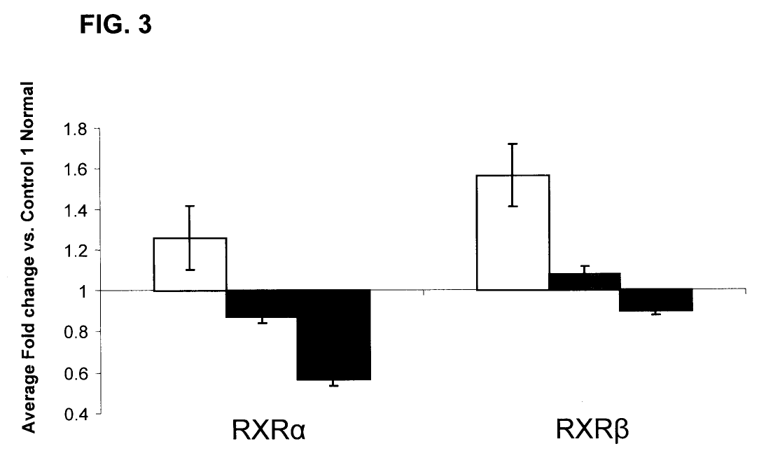

The differences in expression of RXRα and RXRβ in non-lesional and lesional OA cartilage compared to normal, expressed as fold-change, are shown in FIG. 3. Both RXRα (NR2B1) and RXRβ (NR2B2) were found to be expressed at significantly lower levels in both non-lesional and lesional OA cartilage compared to normal.

### Example 4

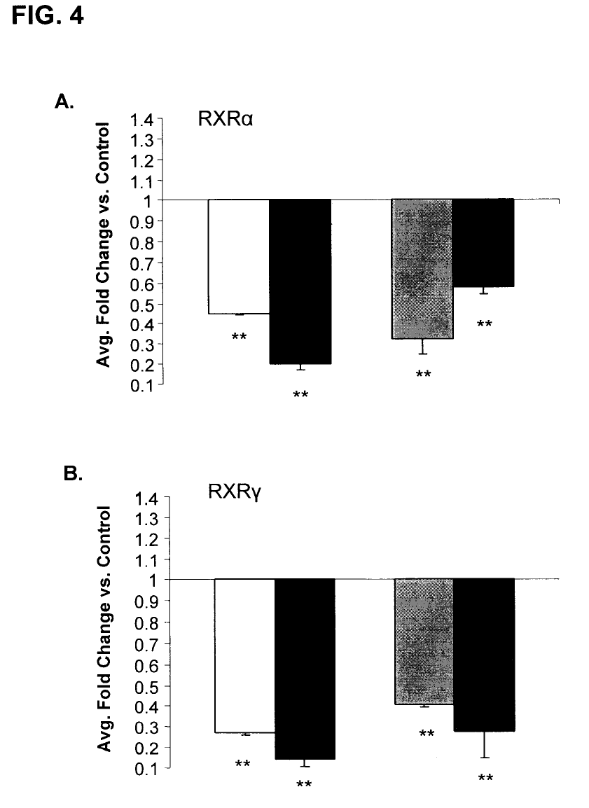

The reduction in RXR expression and its transcriptional activity in OA chondrocytes suggested that it may be under the transcriptional control of signaling pathways that are altered in osteoarthritis. It is well established that IL-1 and TNF are important mediators of OA (Hedbom et al., Cell. Mol. Life Sci. 59:45-53 (2002); Aigner et al., Curr. Opin. Rheumatol. 14:578-84 (2002); Goldring, Arthritis Rheum. 43:1916-26 (2000); Goldring, Curr. Rheumatol. Rep. 2:459-65 (2000)), so experiments were performed to determine whether these cytokines can regulate RXR expression in articular chondrocytes. Primary articular chondrocytes were isolated from human donors (n=4) and grown in monolayer cultures with IL-1β, TNFα, or vehicle, and the expression of RXR isoforms was measured by qRT-PCR. The results of these experiments showed that RXRα expression was significantly reduced by IL-1 (but not TNF) in articular chondrocytes from all donors (FIG. 4A), and RXRγ expression was significantly reduced by TNF treatment (but not IL-1) in articular chondrocytes from all donors (FIG. 4B).

What is claimed is:

1. A method for the treatment of a mammal suffering from osteoarthritis comprising administering to the mammal in need thereof an RXR-responsive gene expression-modulating amount of an RXR agonist.

2. The method of claim 1, wherein the RXR agonist is 9-cis retinoic acid, docosahexanoic acid, phytanic acid, 6-[1-(3,5,5,8,8-pentamethyl-5,6,7,8-tetrahydronaphthalen-2-yl)cyclopropyl]pyridine-3-carboxylic acid, or 4-[1-(5,6,7,8-tetrahydro-3,5,5,8,8-pentamethyl-2-naphthalenyl)ethenyl]benzoic acid.

3. The method of claim 1, wherein treatment with the RXR agonist inhibits cartilage degradation and/or induces cartilage regeneration.

4. The method of claim 1, wherein treatment with the RXR agonist provides pain relief in osteoarthritic joints.

5. A method for the treatment of a mammal suffering from osteoarthritis comprising administering to the mammal in need thereof an effective amount of an RXR agonist to relieve pain in osteoarthritic joints.

6. A method of indentifying an RXR ligand capable of reducing an osteoarthritic effect in cartilage comprising:
(a) providing a sample containing RXR;
(b) contacting the sample with a test compound; and
(c) determining whether the test compound reduces an osteoarthritic effect in cartilage.
